(19) United States
(12) Patent Application Publication
(10) Pub. No.: US 2013/0274598 A1
(43) Pub. Date: Oct. 17, 2013

(54) ILLUMINATION/VIBRATION DEVICE AND FACIAL SKIN MARKING SYSTEM FOR SINUS SURGICAL PROCEDURE

(71) Applicant: Wade W. Han, Windermere, FL (US)

(72) Inventor: Wade W. Han, Windermere, FL (US)

(21) Appl. No.: 13/767,872

(22) Filed: Feb. 14, 2013

Related U.S. Application Data
(60) Provisional application No. 61/598,891, filed on Feb. 14, 2012.

(51) Int. Cl. A61B 5/00

(52) U.S. Cl. CPC A61B 5/0084
USPC 600/427; 600/476; 600/473; 600/437

(57) ABSTRACT

In accordance with at least some embodiments of the present disclosure, a medical device for aiding a medical procedure is disclosed. The medical device contains a vibratory component configured to generate vibrations. The medical device also contains an illumination component configured to generate lights. The medical device further contains a contact component coupled with the vibratory component and the illumination component, wherein the contact component is adapted to maintain contact with a skin area, transmit the vibrations to the skin area, and direct the lights to the skin area.

### CROSS REFERENCE TO RELATED APPLICATION

The present application is related to and claims the benefit of priority of the following commonly-owned, presently-pending provisional application: application Ser. No. 61598891, filed Feb. 14, 2012, entitled “Novel Methods of Identification and Verification of Frontal and Maxillary Sinuses Using External Radio-Opaque Markers on Computed Tomography Imaging, External and Internal Light Trans-illumination as well as Ultrasonic Vibratory Detection and Anesthesia”, of which the present application is a non-provisional application thereof. The disclosures of the forgoing application are hereby incorporated by reference in it entirely, including any appendices or attachments thereof, for all purposes.

### BACKGROUND OF THE DISCLOSURE

Unless otherwise indicated herein, the approaches described in this section are not prior art to the claims in this application and are not admitted to be prior art by inclusion in this section.

Obstruction of frontal sinus outflow pathway is a common cause of chronic frontal sinusitis and headache. The frontal sinus drainage outflows into frontal recess within the anterior ethmoid area, which is complex, tortuous, and buried deep amongst ethmoid cells and recesses. So do the areas behind and above agger nasi, uncinate process and lateral to middle turbinate. Thus, it is extremely difficult to reach and visualize the frontal recess and frontal sinus cavity without removal of some anterior ethmoid sinus cells, even with the patient under anesthesia.

Conventionally, identification of frontal sinus cavity opening and its outflow in its natural form is accomplished by blind probing with a guide wire or surgical instrument.

In other words, the position of the frontal sinus cavity is inconsistently determined by tactile feedbacks received from the guide wire or surgical instrument, when the frontal sinus cavity is trans-illuminated by light from an illumination tip of the guide wire or surgical instrument and the sinus cavity can be seen through thick frontal bone and soft tissue. This method is often inaccurate, as the tip of lighted guide wire may be hanged up in an anterior ethmoid sinus recess during probing.

In other situations, a cell such as interfrontal septal, frontobulla, or type III Kuhn cells may be mistaken as the frontal sinus cavity when the tip of the guide wire or surgical instrument reaches those false frontal spaces which appear indistinguishable to the naked eyes as the true frontal sinus cavity. Also, the position of the tip of the guide wire or surgical instrument within the outflow pathway often can't be determined with this trans-illumination method, due to the distance between the illumination tip and the forehead skin being uncertain.

Alternatively, CT (computed tomography) can be used to determine the frontal sinus outflow pathway in preparation to a frontal sinus visual examination or procedure. However, without triplanar CT image reconstruction and navigation system which are often only available during a frontal sinus surgery under general anesthesia, it is difficult in matching the preoperative CT images with the actual endoscopic localization of the frontal sinus cavity during an in-office sinus procedure.

Pain control is paramount during any awake in-office sinus procedures. Despite adequate topical and local anesthetic applications, pain is often felt during deep sinus instrumentation, where anesthetic spray and injection cannot reach or penetrate the sinus cavity. Patient intolerance due to deep sinus pain is the main reason to abort or terminate awake sinus procedures prematurely.

### BRIEF DESCRIPTION OF THE DRAWINGS

FIG. 1 shows multiple views of a medical device suitable for aiding a sinus surgical procedure;

FIG. 2 shows internal structures of a medical device;

FIG. 3 shows a radio-opaque marking system;

FIG. 4 shows multiple scenarios of using a medical device during a sinus surgical procedure; and

FIG. 5 shows a flow diagram of an illustrative embodiment of a process for aiding a sinus surgical procedure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated herein.

This disclosure is drawn, inter alia, to a medical device and methods for aiding a sinus medical procedure. The sinus medical procedure (also known as sinus surgical procedure) may include, but not limited to, intranasal endoscopic frontal, maxillary sinus outflow dilation with balloon catheters, disease tissue, polyps or tumor removal from frontal or maxillary sinuses, as well as irrigation, suction from and medication delivery to frontal or maxillary sinuses. In some embodiments, a handheld medical device may generate vibrations in ultrasonic frequency range for anesthesia purposes. The handheld medical device may further generate high intensity LED lights and direct the lights onto a patient's facial skin, so that the lights may further propagate, through skin, soft tissue, and bone, into sinus cavities of the patient. This illumination of the sinus cavities may aid an intranasal endoscope in locating these sinus cavities. An ultrasonic probe, microphone, or detector designed for intranasal purposes may also be used along with the nasal endoscope to assist localizing the sinus cavities.

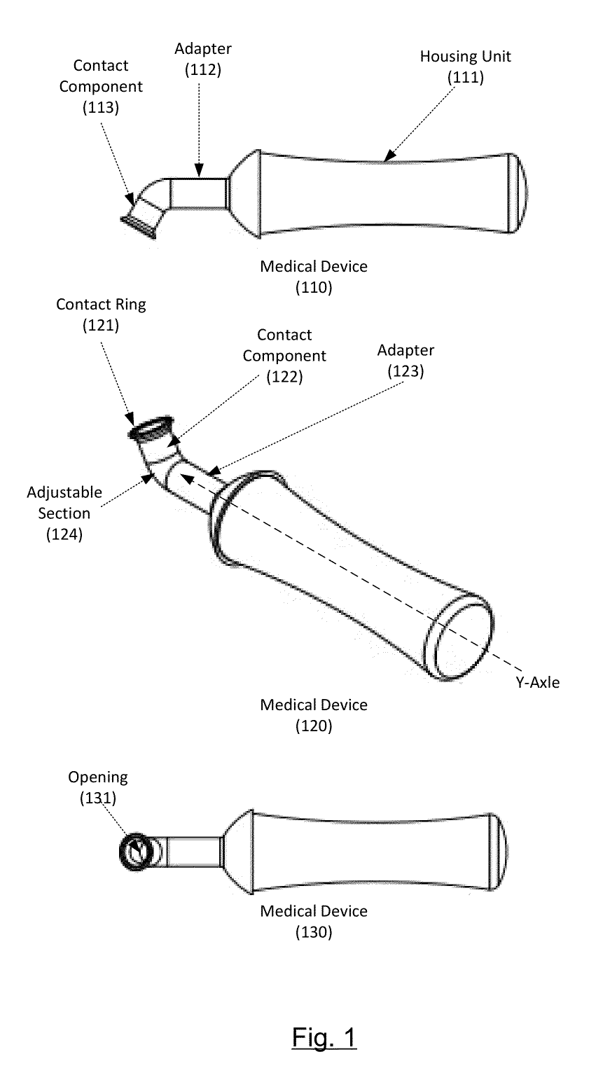

FIG. 1 shows multiple views of a medical device suitable for aiding a sinus surgical procedure, according to certain embodiments of the present disclosure. In FIG. 1, medical devices 110, 120, and 130 represent views from different angles of an embodiment of the medical device. The medical device 110 may contain, among other things, a housing unit 111, an adapter 112, and a contact component 113. The housing unit 111 may be used as a hand-holding place for picking up and grabbing the medical device 110 during operation. The housing unit 111 may contain one or more electronic components for generating vibrations and/or lights. The details of the electronic components contained in the housing unit 111 are further described below.

In some embodiments, the adapter 112 may be configured to connect the contact component 113 with the housing unit 111. In other words, the adapter 112 may have a first end connected with the housing unit 111, and a second end connected with the contact component 113. The adapter 112 may be configured to transmit the vibrations generated by one of the electronic components in the housing unit 111 to the contact component 113. Further, the lights generated by another one of the electronic components in the housing unit 111 may also be transmitted to the contact component 113 through the adapter 112. The adapter 112 may further be configured to adjust the contact component 113, as described below.

In some embodiments, the contact component 113 may be configured to maintain contact with a skin area of a patient undertaking the sinus surgical procedure. In other words, the contact component 113 may have a third end connected with the second end of the adapter 112, and a fourth end (open end) configured to maintain contain with the skin area. During operating, an operator may hold the housing unit 111, and place the open end of the contact component 113 against a specific area on the patient's skin (e.g., forehead skin area or cheek facial skin area). The operator may apply some pressures to the housing unit 111, which in turn may transfer the pressures, through the adapter 112 and the contact component 113, to the skin area of the patient. To ensure the medical device 110 functioning properly, the open end of the contact component 113 may be firmly in contact with the patient's skin area throughout the whole sinus medical process.

In some embodiments, the medical device 120 may have a contact component 122 (which is similar to the contact component 113 of the medical device 110) and an adapter 123 (which is similar to the adapter 112 of the medical device 110). The contact component 122 may be further configured with a contact ring 121 located at its open end. The contact ring 121, which may be made of a soft or semi-soft material such as rubber or plastic, may be configured to enclose a section of the skin area and provide a tight and sealed contact between the contact component 122 and the section of the skin area. The contact ring 121 can also reduce/distribute some of the pressures applied to the skin area from the medical device 120, thereby minimizing any pain or discomfort the patient's might feel during the sinus surgical procedure.

In some embodiments, the adapter 123 of the medical device 120 may optionally have an adjustable section 124 that is located at the end that connects with the contact component 122. The adjustable section 124 may allow the contact component 122 to be adjusted to a certain angle with respect to the adapter 123 and the housing unit that is attached to the adapter 123. In other words, the contact component 122 may be adjusted to any degree (from approximately 0 degree to approximately 90 degree in relationship to the rest part of the medical device 120) by bending the medical device 120 at the adjustable section 124. Further, the adjustable section 124 may allow the contact component 122 to rotate in a 360-degree range around a Y-axle of the medical device 120. In other words, the adjustable section 124 may have a concertina-like structure allowing it to bend and rotate, in a fashion that is similar to an adjustable section of an articulated beverage straw.

In some embodiments, a view illustrated by the medical device 130 may show an opening 131 at one end of its contact component. The opening 131 may be a cup-like distal tip applicable to a patient's skin area which is above a sinus cavity, such as frontal or maxillary sinus cavity. The opening 131 may allow lights generated from an illumination component of the medical device 130 to pass through and be directed to the skin area. In this case, the adapter, the adjustable part, as well as the contact component may be made of nontransparent or semitransparent, light-shielding materials such as wood, metal, plastic, or rubber. Such an approach may minimize light energy leaking through the bodies of these parts, and maximize the light intensity emitting out of the opening 131 and arriving at the skin area. For example, the adapter, the adjustable part, and the contact component may be made of a latex-free, non-transparent, and hypo-allergenic soft plastic.

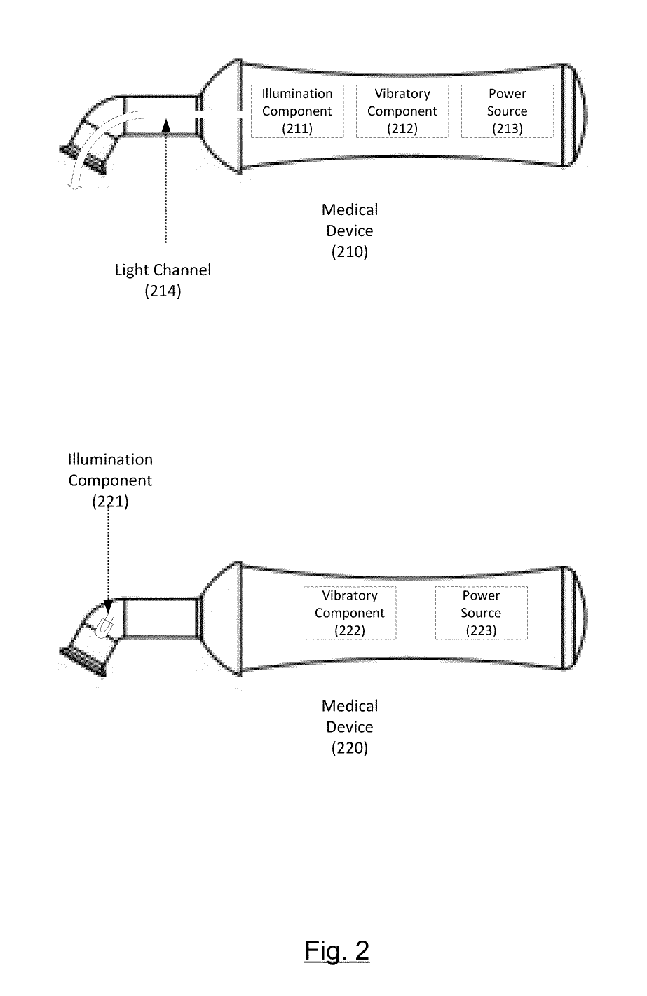

FIG. 2 shows internal structures of a medical device, according to various embodiments of the present disclosure. In FIG. 2, a medical device 210 (which is similar to the medical devices 110, 120, and 130 of FIG. 1) contains, among other things, an illumination component 211, a vibration component 212, and a power source 213. The power source 213 may be a battery which can supply sufficient electric powers for the operating of the medical device 210. The medical device 210 may further contain a power switch (not shown in FIG. 2) for the turning on and off of the power source 213, the vibration component 212, and/or the illumination component 211, individually or in combination.

In some embodiments, the illumination component 211 may be a high intensive LED light bulb that can emit lights that has sufficient intensity to pass through multiple layers of facial skin, soft tissue, and/or bone. Specifically, the illumination component 211 may generate human visible lights which may penetrate into the sinus cavities of a patient, and be observed by human eyes when viewing through an intranasal endoscope. Alternatively, the illumination component 211 may emit human-invisible, machine-detectable lights (e.g., infra-red, X-ray, ultrasonic wave, etc) that can be detected in the sinus cavities by an ultrasonic probe, microphone, or any intranasal detector.

In some embodiments, a light channel 214 may be installed in the adapter and the contact component of the medical device 210, in order to transmit the lights generated by the illumination component 211 to the opening of the contact component. The light channel 214 may be an optical mechanism having reflective mirrors or fibre optical cables, allowing the lights to travel through the bended adjustable section of the adapter and the contact component before reaching the skin area that are enclosed by a contact ring of the medical device 210.

In some embodiments, the vibration component 212 may be configured to generate vibrations that are suitable for numbing the nerves innervating the sinus cavities and reducing pains during a sinus surgical procedure. The generated vibrations may be in an infrasonic frequency range or ultrasonic frequency range. Further, the vibration component 212 may generate vibrations having sufficient intensity that can transmit through the adapter and the contact component of the medical device 210, and propagate through the skin, soft tissue, and bone of a patient. In other words, the vibration energy originated from the vibrations may be effective at or around a patient's sinus cavity and/or sinus outflow pathway for anesthesia purposes.

In some embodiments, the medical device 220 may have an illumination component 221 (which is similar to the illumination component 211 of the medical device 210) installed in its contact component, rather than in its housing unit. In this case, the illumination component 221 may be configured to directly illuminate the skin area that is enclosed by the opening of the medical device 220. Such an approach may eliminate the need for a light channel for directing the lights from an illumination component that is located inside of the housing unit of the medical device 220.

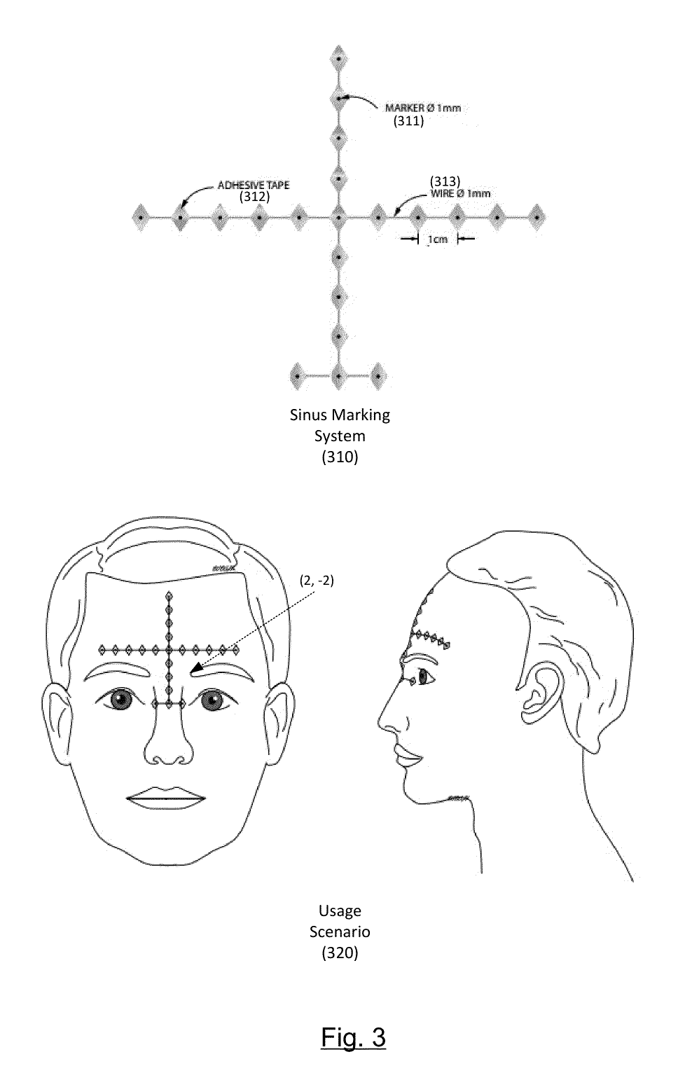

FIG. 3 shows a radio-opaque marking system, according to certain embodiments of the present disclosure. The frontal sinus drainage outflow pathway of a patient may have a tremendous inter-subject variability, and is therefore difficult to visualize and mentally reconstruct based on a conventional orthogonal triplanar CT image analysis. In some embodiments, a radio-opaque marking system 310 may be used to label the forehead and periorbital surface topography of the patient during a sinus CT imaging scanning.

In some embodiments, the radio-opaque marking system 310 may include a set of skin markers 311 which can be used to provide coordinates for referencing the intranasal structure under a patient's facial skin. For example, the sinus marking system 310 may include 21 radio-opaque markers 311 that are linked using 3 radio-opaque wires 313. Each marker 311 may be opaque when observed under a CT image scanning process, and may have a substantially circular shape with a size of about 1 mm in diameter. The wires 313 may also be opaque when viewed by the CT scanning process, and may have a thickness of about 1 mm. The distance between any two of the markers 311 on a specific wire 313 may be about 1 cm. Each marker 311 may have a diamond-shaped latex-free adhesive tape 312 attached, allowing the sinus marking system 310 to be firmly placed on a patient's forehead without slipping.

In some embodiments, as shown in FIG. 3, the radio-opaque marking system 310 may have two horizontal wires 313 (a long horizontal one and a short horizontal one) intersecting with a third vertical wire 313 (which is perpendicular to the two horizontal wires). The long horizontal wire and the vertical wire may form a coordinate system similar to a X-Y coordinate system. The short horizontal one may be used for the proper positioning of the radio-opaque marking system 310 onto the patient's forehead. As shown in the usage scenario 320, the sinus marking system 310 may be put on the forehead of the patient by aligning the lower shorter horizontal wire with the inter-canthal line between the patient's two corners of eyes. Afterward, the vertical wire of the marking system 310 may be placed along the middle line of the patient's face, and the longer horizontal wire may be placed above the patient's eyebrows. Thus, by referencing the specific markers on the sinus marking system 310, a specific location on the forehead or underneath the forehead may be properly identified.

For example, by using a referencing scheme similar to the referencing scheme of the X-Y coordinate, the inner corner of the patient's right eyebrow may be identified as being located at (2, -2). That is, the center marker, which is located at the intersection of the long horizontal wire and the vertical wire, may have a coordinate position of (0.0). Thus, the (2,-2) position is a location which is identify-able based on the second marker on the right of the center marker, and the second marker below the center marker. Further, once being placed onto the patient's forehead, the marking system 310 may become a 3-D marking system. In other words, the marking system 310 can be used to identify a 3-D location inside of the patient's head, in a fashion that is similar to the identification of a position in a X-Y-Z 3-D coordinate system.

Prior to a sinus procedure, after the sinus marking system 310 is placed onto the patient's upper face as shown above, the locations of the frontal sinus cavities as well as the supraorbital and infraorbital nerves may be ascertained from the CT images, and identified using the locations of the specific markers which are clearly visible in the CT images. Afterward, the frontal sinus cavities as well as the supraorbital and infraorbital foramens may be identified using the locations of those markers 311.

Thus, the sinus marking system 310 may guide the optimal placement of the external trans-illumination lights originated from a medical device as shown in FIG. 1. The proper localization of the frontal sinus cavity also helps the surgeon to determine if the internal trans-illumination from the lighted guide wire or instrument is placed in the correct frontal sinus cavity, not in the misleading pockets of variant anterior ethmoid sinus cells such as interfrontal septal, supra-orbital ethmoid, or Type III Kuhn cells. The markers shown on the sinus CT images can also assist the surgeon to locate the supra-orbital and infra-orbital nerve bundles, in order to optimally place the vibratory anesthesia device.

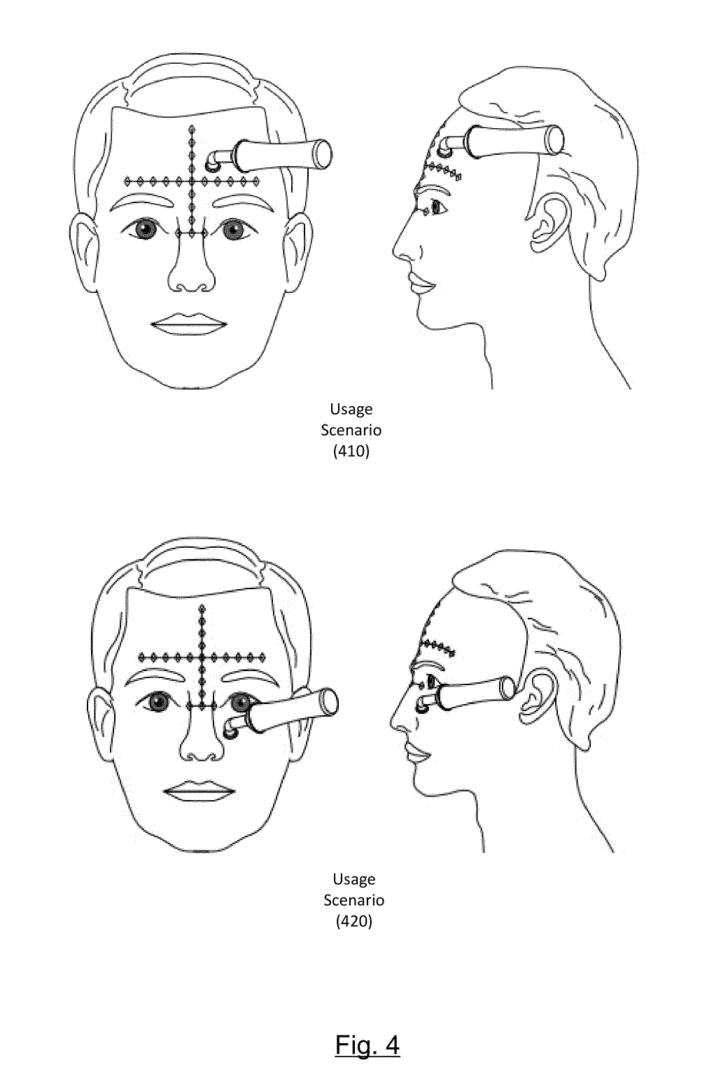

FIG. 4 shows multiple scenarios of using a medical device during a sinus surgical procedure, according to certain embodiments of the present disclosure. A medical device as shown in FIG. 1 may be used during an awake sinus surgical procedure. In usage scenario 410, the medical device may be turned on for its vibration function, and placed at or around the supraorbital foramen of a patient to anesthetize the surrounding supraorbital sensory nerves. In usage scenario 420, the medical device may be placed at or around the infraorbital foramen to anesthetize the surrounding infraorbital sensory nerves of the patient. Thus, the medical device may be used to supplement any local and/or topical anesthesia to the frontal and/or maxillary sinus outflow pathway, respectively.

Further, the medical device may be turned on for its illumination function, and placed at the patient's supraorbital and/or infraorbital foramens. The LED lights emitted from the medical device may assist the visualization and identification of the frontal sinus outflow pathway during intranasal endoscopy procedure. Specifically, following the insertion of a guide wire or instrument with lighted tip into the proposed or assumed frontal sinus cavity, the room light and the light from the tip of the guide wire or instrument may be turned off temporarily. Afterward, the medical device with the turned on LED lights may be placed on the patient's skin areas as shown in the usage scenarios 410 and 420. Thus, by examining the lights that penetrate the skin, soft tissue, and bone and illuminate the frontal sinus cavity, the tip of the guide wire or instrument can be moved to the correct location by looking through the intranasal endoscopy.

In some embodiments, an ultrasonic probe or microphone can be used next to the tip of the endoscope to further ascertain the direction of external ultrasonic transmission. In other words, the medical device may transmit ultrasonic waves instead of LED lights, and may be placed directly above the frontal sinus cavity. Thus, when the ultrasonic probe or microphone detects the direction of the ultrasonic waves coming from a certain direction (e.g., directly above), the location of the frontal sinus cavity or resonance chamber may also be ascertained.

In some embodiments, with the assistance of a sinus marking system as shown in FIG. 3, the above verification process may be further improved for better accuracy. Under the anterior table of the frontal bone and forehead soft tissue of a patient, there are several competing air spaces in addition to the frontal sinus cavity. During a sinus surgical procedure, the internal lighting tip of a guide wire or instrument may be placed in the potentially false sinus spaces such as interfrontal septal cell, supraorbital ethmoid cell, type III Kuhn cell, or an extensive superior anterior terminal recess of middle meatus or infundibulum (Recess Terminalis). The sinus marking system may greatly improve the surgeon's ability to differentiate those potentially false sinus spaces from the true frontal sinus cavity.

In some embodiments, the sinus marking system may first be placed on the patient's forehead as illustrated above. Afterward, a pre-procedure CT image may be taken for the patient's frontal sinus area. The CT image, which has the opaque markers and wires shown, may be analyzed for identifying the true frontal sinus cavity. Specifically, the markers in the sinus marking system may be used as a coordinate system to pinpoint the location of the sinus cavity from the patient's frontal face perspective.

During a subsequent sinus surgical procedure, the medical device may be placed at the coordinate which is previously identified, so that lights emitted from the medical device may be able to penetrate the skin, soft tissue, and bone, and directly illuminate the sinus cavity. Thus, the intranasal endoscopy may be able to correctly locate the true sinus cavity by seeking out the lights from the medical device. Further, the coordinate may be patient specific, and may be used repeatedly for the seeking of the sinus cavity during any future sinus surgical procedures.

In some embodiments, once the lighted tip of the guide wire or instrument is located in a sinus cavity, the lights on the intranasal endoscopy may be turned off, leaving the lights emitted from the tip of the guide wire or instrument on. Thus, a light spot originated from the tip of the guide wire or instrument may be shown on the patient's frontal forehead or cheeks through the bone, soft tissue, and skin. Since the sinus marking system may still be placed on the patient's face or be newly placed onto the patient's face in an identical fashion, the light spot shown on the patient's forehead or cheek may then be recorded as a “current coordinate” of the tip of the guide wire or instrument. The current coordinate may then be compared with a “previous coordinate” identified from the CT scan images and associated with the known true sinus cavity. If the current and previous coordinates are identical or sufficiently close enough, then the surgeon may be certain that the tip of the guide wire or instrument is in the true sinus cavity. Otherwise, the medical device may be placed on the forehead location identified by the previous coordinate, and the guide wire or instrument may be adjusted accordingly in order to reach the true sinus cavity. If the current coordinate if found to be more accurate than the previous coordinate, the current coordinate may replace the previous coordinate and be used for the seeking of the sinus cavity during any future sinus surgical procedures.

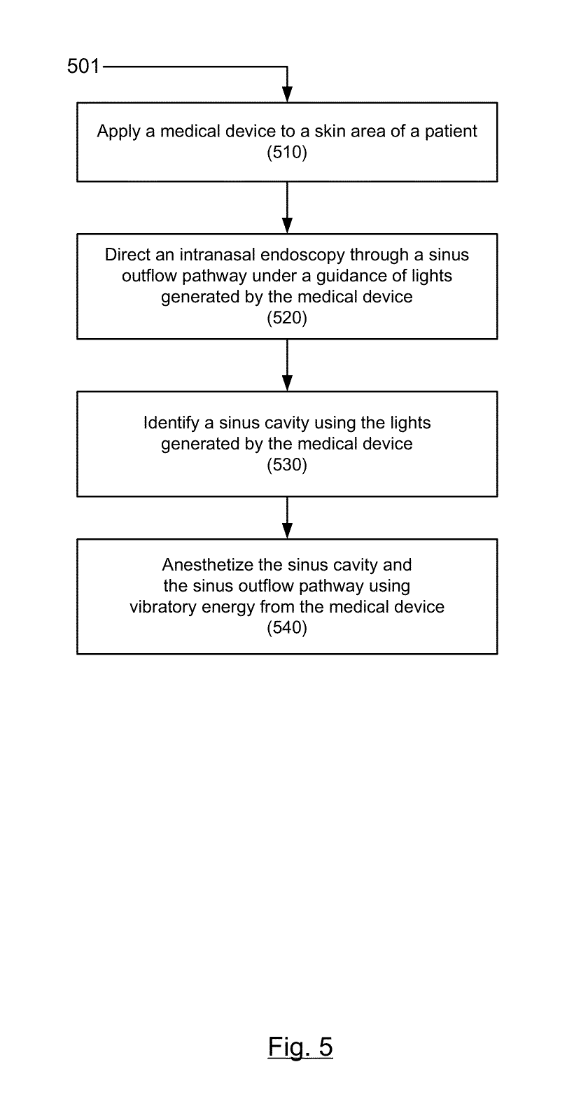

FIG. 5 shows a flow diagram of an illustrative embodiment of a process 501 for aiding a sinus surgical procedure. The process 501 sets forth various functional blocks or actions that may be described as processing steps, functional operations, events, and/or acts, which may be performed by hardware, software, and/or firmware. Those skilled in the art in light of the present disclosure will recognize that numerous alternatives to the functional blocks shown in FIG. 5 may be practiced in various implementations.

In some embodiments, a cavity detecting system may be configured to perform the process 501 as described below. The cavity detecting system may include a medical device such as the medical device shown in FIG. 1. The cavity detecting system may also include mechanisms that can control and operate the medical device, the guide wire and instrument, the intranasal endoscopy, and/or other medical devices used during a sinus surgical procedure. Further, the cavity detecting system may be controlled manually by an operator or automatically by a software program or another system.

At block 510, the cavity detecting system may apply a medical device (e.g., a vibration and illumination device) to a skin area of a patient. In some embodiments, the medical device may contain a vibratory component configured to generate vibrations, an illumination component configured to emit lights, and a contact component adapted to maintain contact with the skin area, transmit the vibrations to the skin area and direct the lights to the skin area.

At block 520, the cavity detecting system may direct an intranasal endoscopy through the sinus outflow pathway under a guidance of the lights generated by the medical device during an awake sinus procedure. Specifically, the medical device may be turned on, and the cavity detecting system may detect the lights generated by the medical device intra-nasally.

At block 530, the cavity detecting system may identify a sinus outflow pathway outlet using the lights penetrated through the skin area. In some embodiments, the cavity detecting system may turn off a light located at a tip of the intranasal endoscopy. Afterward, the cavity detecting system may detecting the lights generated by the medical device using the intranasal endoscopy or other light detecting instruments located intra-nasally. Further, the cavity detecting system may direct an intranasal endoscopic instrumentation for sinus dilation, tissue removal, or irrigation through the sinus outflow pathway under the guidance of the lights generated by the medical device. The cavity detecting system may also direct an intranasal drug delivery into the sinus cavity under the guidance of the lights generated by the medical device.

In some embodiments, the cavity detecting system may identify a location of the sinus cavity and the sinus outflow pathway using a probe which is configured to ascertain a direction of the vibrations. In other words, the medical device may be placed directly above or nearby a sinus cavity or sinus outflow pathway. The intranasal endoscopy, a probe, or a similar instrument may have a detector located at its tip and configured to detect a direction of the lights or vibrations generated by the medical device. Based on the detected direction, the location of the sinus cavity or sinus outflow pathway may also be ascertained.

In some embodiments, the cavity detecting system may optionally apply a sinus marking system to the skin area. In some embodiments, the sinus marking system may contain a plurality of radio-opaque markers and a plurality of radio-opaque wires. Afterward, the cavity detecting system may take a CT scan image of the skin area. The CT scan image may include the plurality of radio-opaque markers and the plurality of radio-opaque wires. Thus, the cavity detecting system may identify the sinus cavity or the sinus outflow pathway based on the CT scan image based on the locations of the plurality of radio-opaque markers and the plurality of radio-opaque wires.

At block 540, the cavity detecting system may anesthetize the patient's sinus cavity or the sinus outflow pathway using vibratory energy originated from the vibrations generated from the medical device and transmitted through the skin area. In some embodiments, the cavity detecting system may place the medical device to the skin area surrounding supraorbital foramen or infraorbital foramen, and turn on the medical device, thereby allowing the vibrations generated by the medical device to anesthetize supraorbital or infraorbital sensory nerves.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments. Moreover, one or more of the outlined steps and operations may be performed in parallel.

Thus, methods and systems for a medical device have been described. Although the present disclosure has been described with reference to specific exemplary embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense.

I claim:

1. A medical device for aiding a medical procedure, comprising:
a vibratory component configured to generate vibrations;
an illumination component configured to generate lights; and
a contact component coupled with the vibratory component and the illumination component, wherein the contact component is adapted to maintain contact with a skin area, transmit the vibrations to the skin area, and direct the lights to the skin area.

2. The medical device as recited in claim 1, further comprising:
a housing unit configured to house the vibratory component and the illumination component; and
an adapter for connecting the housing unit with the contact component, wherein the adapter is configured to transmit the vibration from the vibratory component to the contact component, and the adapter is further configured to adjust an angel between the housing unit and the contact component.

3. The medical device as recited in claim 2, wherein the adapter contains a light channel allowing the lights generated from the illumination component to be transmitted through the light channel to an opening on the contact component.

4. The medical device as recited in claim 1, wherein the illumination component is a LED light, an infra-red light, or an ultrasonic wave generator.

5. The medical device as recited in claim 1, wherein the vibration component is configured to generate the vibrations in an infrasonic or ultrasonic frequency range.

6. The medical device as recited in claim 1, further comprising:
a housing unit configured to house the vibratory component; and
an adapter for connecting the housing unit with the contact component, wherein the adapter is configured to transmit the vibrations from the vibratory component to the contact component, and the contact component is configured to house the illumination component.

7. The medical device as recited in claim 1, further comprising:
a power source configured to provide electricity to the vibratory component and the illumination component; and
a switch configured turn on and turn off the power source, the vibration component, or the illumination component.

8. The medical device as recited in claim 1, wherein the contact component further comprises a contact ring located at a first end of the contact component and configured for maintaining the contact with the skin area.

9. A medical device for aiding a medical procedure, comprising:
a housing unit containing a vibratory component configured to generate vibrations and an illumination component configured to generate lights;
an adapter having a first end connected with the housing unit, a second end and a light channel, wherein the adapter is configured to transmit the vibrations from its first end to its second end, and the lights from its first end to its second end via its light channel; and
a contact component having a third end and a fourth end, wherein the third end of the contact component is connected with the second end of the adapter, the fourth end is configured to maintain contact with a skin area, and the contact component is configured to transmit the vibrations from its third end to its fourth end, and the lights from its third end to its fourth end.

10. The medical device as recited in claim 9, wherein the contact component further comprises a contact ring located at a first end of the contact component and configured for maintaining the contact with the skin area.

11. The medical device as recited in claim 9, wherein the adapter is further configured to adjust an angel between the housing unit and the contact component.

12. The medical device as recited in claim 9, wherein the illumination component is a LED light, an infra-red light, or an ultrasonic wave generator.

13. The medical device as recited in claim 9, wherein the vibration component is configured to generate the vibrations in an infrasonic or ultrasonic frequency range.

14. A method for aiding a medical procedure, comprising:
applying a medical device to a skin area, wherein the medical device contains a vibratory component configured to generate vibrations, an illumination component configured to emit lights, and a contact component adapted to maintain contact with the skin area, transmit the vibrations to the skin area, and direct the lights to the skin area; and
identifying a sinus cavity or a sinus outflow pathway outlet using the lights penetrated through the skin area.

15. The method of claim 14, further comprising:
applying a sinus marking system to the skin area, wherein the sinus marking system contains a plurality of radio-opaque markers and a plurality of radio-opaque wires;
taking a CT scan image of the skin area, wherein the plurality of radio-opaque markers and the plurality of radio-opaque wires are shown in the CT scan image; and
identifying the sinus cavity or the sinus outflow pathway based on the CT scan image by identifying locations of the plurality of radio-opaque markers and the plurality of radio-opaque wires.

16. The method of claim 14, wherein the identifying of the sinus outflow pathway comprising:
directing an intranasal endoscopy through the sinus outflow pathway under a guidance of the lights generated by the medical device during an awake sinus procedure;
directing an intranasal endoscopic instrumentation for sinus dilation, tissue removal, or irrigation through the sinus outflow pathway under the guidance of the lights generated by the medical device; and
directing an intranasal drug delivery into the sinus cavity under the guidance of the lights generated by the medical device.

17. The method of claim 16, wherein the identifying of the sinus outflow pathway further comprising:
turning off a light located at a tip of the intranasal endoscopy; and
detecting the lights generated by the medical device using the intranasal endoscopy located intra-nasally.

18. The method of claim 14, further comprising:
anesthetizing the sinus cavity or the sinus outflow pathway using vibratory energy originated from the vibrations transmitted through the skin area.

19. The method of claim 18, wherein the anesthetizing of the sinus cavity comprising:
placing the medical device to the skin area surrounding supraorbital foramen or infraorbital foramen; and
turning on the medical device, allowing the vibrations generated by the medical device to anesthetize supraorbital or infraorbital sensory nerves.

20. The method of claim 14, further comprising:
identifying a location of the sinus cavity and the sinus outflow pathway using a probe which is configured to ascertain a direction of the vibrations.
